(19) United States
(12) Patent Application Publication
(10) Pub. No.: US 2011/0208063 A1
(43) Pub. Date: Aug. 25, 2011

(54) NON-CONTACT FREQUENCY DOMAIN NEAR INFRARED ABSORPTION (fNIR) DEVICE FOR ASSESSING TISSUE DAMAGE

(75) Inventors: Elisabeth S. Papazoglou, Yardley, PA (US); Leonid Zubkov, Philadelphia, PA (US)

(73) Assignee: DREXEL UNIVERSITY, Philadelphia, PA (US)

(21) Appl. No.: 13/125,116

(22) PCT Filed: Nov. 6, 2009
(86) PCT No.: PCT/US09/63564

§ 371 Date: May 10, 2011

Related U.S. Application Data
(60) Provisional application No. 61/111,924, filed on Nov. 6, 2008.

(51) Int. Cl. A61B 6/00

(52) U.S. Cl. 600/473

(57) ABSTRACT

A device for measuring the progress of healing of a wound over time includes at least one diode laser source that provides respective input wavelengths into at least one source fiber, a first optical switch that sequentially switches wavelengths among the respective input wavelengths into the at least one source fiber, a probe that does not touch the wound dunng use, the probe including the at least one source fiber and at least two detectors spaced thereon, an optical system that provides source light from the at least one source fiber to the wound and that detects light scattered by the wound surface and provides the scattered light to the detectors, and a processing unit responsive to outputs of the detectors for providing at least four independent measurements for calculation of an absorption coefficient/Ia and a scattering coefficient/I's of the light by the wound

### CROSS-REFERENCE TO RELATED APPLICATIONS

The present application claims priority to U.S. Provisional Patent Application No. 61/111,924, filed Nov. 6, 2008. The contents of that application are hereby incorporated by reference.

### FIELD OF THE INVENTION

The present invention relates to a frequency domain near infrared absorption device for assessing tissue damage in a wound without physically contacting the wound.

### BACKGROUND OF THE INVENTION

A variety of instruments based on the diffuse propagation of Near Infrared (NIR) photons due to multiply scattered light have been used in the prior art to obtain clinically meaningful information about living tissue, such as tissue oxygenation. Such devices rely on optical fibers to transport the incident and scattered lights; however, the fiber optical probe is in contact with the tissue under examination. One of the key advantages of these laser technologies is their non-invasive nature; however, this advantage is negated by the fact that the fiber optical probe contacts the tissue under examination.

The present inventors have previously described in an article entitled “Optical Properties of Wounds: Diabetic Versus Healthy Tissue,” IEEE Transactions on Biomedical Engineering, Vol. 53(6), pages 1047-1055, June, 2006, such a frequency domain NIR instrument with one source position, four detector channels, three wavelength diode lasers (λ=685 nm, 785 nm and 830 nm) and a source modulation frequency of 70 MHz. Such a device has been demonstrated by the inventors as useful in assessing the early healing process of wounds in healthy and diabetic animals. For example, the device incorporates the ability to assess the extent of hydration at the wound site in addition to the detection of oxygenated, deoxygenated hemoglobin and amount of blood. The wound healing assessment is further enhanced with the added capability to vary sensor penetration depth by adjusting the probe design. As illustrated in FIG. 1, the device included a 70 MHz RF signal with stable phase provided by RF generator block 1. The 70 MHz signal supplied the IQ demodulators of the four detectors 6-9 and modulated the emission light of three laser diodes 2. Two 1×2 optical switches and their drivers for optical switch 3 are were controlled using software executed on the illustrated personal computer 11 with control digital signals 12 being provided via data acquisition board (DAQ) 10. Light from the optical switch 3 was fed to 62.5 micron source fiber inserted in a probe 4 that was used to illuminate the experimental animal 5, one wavelength at a time. The scattering light was picked up by the four IQ detector bundle fibers 6-9. The detected electrical RF signal was then amplified by a first amplifier, filtered by a bandpass filter at 70 MHz, further amplified by a second amplifier, and fed to an I/Q demodulator (not shown). The outputs of the IQ detectors 6-9 were the cosine (I) and sine (Q) low frequency components of amplitude and phase shift relative to the reference signal from the RF generator 1. These signals were further digitized by a 16-bit DAQ 10 for processing by the computer 11.

However, the device of FIG. 1 collects the light scattered from the tissue site using optical fibers that are in contact with the tissue surface and transfer this light to optical detectors 6-9. Such contact with the wound is undesirable and the contact probe provides no flexibility in rearranging the detector fibers and optimizing the setup for wounds of different surface areas, shapes, and depths because one source and four detector fibers are permanently inserted in a rectangular piece made of plastic, Teflon, or silicon.

Leonardi et al. describe in US 2006/0155193 another method for using a near infrared spectroscopy device to assess burn injuries. Leonardi et al. purport to use broadband white light and measure the intensity of the reflected light using a CCD. However, this device cannot obtain absolute values of absorption scattering coefficients but instead obtains relative changes. Moreover, the probe also must penetrate into the burned skin, which is generally undesirable.

There are many medical applications where it would be preferable or necessary not to touch the injury or wound site. Since the principle of operation of an fNIR device is to register the light scattered from the tissue, contact is not a limiting factor for the success so long as the light may be captured and its origin in the tissue accurately tracked. The present invention is directed to such a non-contact device.

Those skilled in the art will appreciate that in the field of NIR devices, the non-contact Continuous Wave (CW) method has been used for rapid and accurate acquisition of large data sets of tissue optical properties to reconstruct 2D or 3D images, for example, in breast imaging to detect tumors. Depending on the desired tissue volume to be covered (usually around 1 liter) and the required resolution (typically 0.2-1.0 cm), a very large number of measurements (from 10³ to 10⁵) are needed. A CCD camera is the most common device for reconstruction and is coupled with high quality lenses to achieve coverage of a substantial volume of tissue instead of an experimental probe with fibers, thus allowing for continuous wave (CW) measurements.

A frequency domain NIR device with a remote probe has been implemented for image reconstruction work and is based on a CCD camera coupled to a gain-modulated image intensifier with Fast Fourier Transform. This device is described in an article by Godavarty et al. entitled “Fluorescence-enhanced optical imaging in large tissue volumes using a gain-modulated ICCD camera,” Physics in Medicine and Biology, Vol. 48, pages 1701-1720 (2003), and in an article by Gurfinkel et al. entitled “Determination or optical properties in semi-infinite turbid media using imaging measurements of frequency-domain photon migration obtained with an intensified charge-coupled device,” J. of Biomedical Optics, Vol. 9, pages 1336-1346 (2004). However, this device is very expensive and must be used on an optical table with very stable temperature and humidity conditions. As such, it is not suitable for clinical use.

A non-contact device having the same sensitivity and improved robustness compared to devices that must contact the wound is desired for many reasons. For example, a non-contact device may be used to obtain data from practically any wound and burn and, by not touching the injured skin, the measurements do not cause any pain or contamination of the wound. Other benefits of a non-contact device include the ability to maintain a sterile environment within measurements without a need for intermediate steps for sterilization, the elimination of operator variability due to differing contact pressures, and the ability to obtain measurements faster by a single operator. A non-contact device also may be mounted on a hyperbaric oxygen chamber for monitoring the status of a wound during and after treatments.

Several different types of frequency domain near infrared (fNIR) devices are known in the art. Currently three major experimental methods are used in the NIR range to measure absorption and scattering coefficients μ_{α} and μ′ₛ in multiply scattering tissues. The key difference among the various techniques lies in the source of incident light. The simplest and easiest method uses constant power lasers as, for example, in continuous wave (CW) devices. Since the power source for this method is constant, it is only possible to measure one parameter, the intensity of scattered light. Changes in this light intensity are measured as a function of source-detector separation ρ. In the case of CW devices, however, difficulties emerge when trying to separate absorption attenuation from scattering effects. CW methods give a composite “picture” of light intensity changes and cannot distinguish scattering from absorption. It is however possible to distinguish scattering from absorption phenomena using the following equation:

$$φ ( r , t ) = \frac{{vM}_{D}}{4 π Dr} exp ( i kr ) exp ( - iω t ) , \qquad ( 1 )$$

Equation (1) represents a solution of the diffusion equation for infinite homogeneous highly scattering media where Φ(r, t) is the photon fluence, ν is the speed of light in turbid medium, D=ν/3μ′ₛ is the photon diffusion coefficient, μ′ₛ=μₛ(1−g) is the reduced scattering coefficient, g=<cos θ> is the mean cosine of the photon scattering angle, and μₛ is the reciprocal of the scattering length. The complex diffuse wave wavenumber is a very important parameter=kᵣ+kₜ. The square of the wave number k²=(−3μ_{α}μ′ₛ+iωtμ′ₛ) is an expression where both coefficients μ_{α} and μ′ₛ. are represented in an almost symmetrical expression and contribute in a similar way on the measured photon fluence Φ(r,t).

Both time resolution spectroscopy and the frequency domain technique are able to determine μ_{α} and μ′ₛ simultaneously. In the case of time resolved spectroscopy, a sequence of very short light pulses falls on the tissue under investigation and the broadening and shape of light impulses scattered from the tissue is analyzed. Although a wealth of information can be obtained, this method is complex and expensive and is difficult to implement in a routine clinical setting.

Time resolution spectroscopy (TRS) instruments also are able to obtain high quality information about the optical properties of the tissue from the broadening of very short light pulses after their propagation in tissue. Although rich in information obtained, this method is complex and expensive and difficult to implement in a routine clinical setting.

The frequency domain technique with single modulation radio frequency RF of the incident light and variable source-detector separations can be used to simultaneously assess μₛ′ and μ_{α} of tissue, with a simpler and more cost effective device. Frequency domain devices measure directly two parameters: a) the intensity I(r,t) of scattered light, as in the case of CW methods, and b) the value of the phase shift Δφ, a parameter not obtained in CW methods. The phase shift is a result of the light modulation in that there is a shift between the RF of modulated scattered light compared to the phase of the RF oscillator which is used for modulation. The phase shift Δφ occurs because of the diffusive aspects of light propagation in tissue representing multiple light scattering phenomena. For typical optical measurements where light enters the tissue through the skin and leaves the tissue at distance ρ from the entry point, the real path of light R in the tissue is R˜(10-20)*ρ by reason of the diffusion propagation of light. Fitting the experimental values of intensity and phase shift to the solution of the diffusion equation allows simultaneous determination of μₛ′ and μ_{α} in tissues. For these reasons, the inventors believe that the frequency domain technique will provide the most suitable non-contact device.

### SUMMARY OF THE INVENTION

The invention provides a system for providing non-contact measurements of wounds using a frequency domain NIR device. In particular, a frequency domain technique with single modulation frequency and variable source-detector separations is used to calculate μₛ′ and μ_{α} from the surface of wounds and tissues in vivo. The in vitro calibration of the device and the semi-infinite medium approximation to the diffusion equation then may be employed to extract optical coefficients from amplitude and phase measurements. In turn, the progress of the healing of the wound may be determined from the values of these optical coefficients.

A frequency domain modification of an fNIR device calculates tissue optical properties (scattering coefficient μₛ′ and absorption μ_{α}) from the measured amplitude and phase of scattered light. The amplitude of the diode laser radiation is modulated at RF frequencies. Measured data shows that, in the NIR region, the change of the absorption coefficient μ_{α} reflects the variation in oxygenated and deoxygenated hemoglobin concentration because hemoglobin is the main absorption chromophore in the wavelength range 680-850 nm along with water and lipids.

A device for measuring the progress of healing of a wound over time in accordance with a first embodiment of the invention includes at least one diode laser source that provides respective input wavelengths into one of at least two source fibers, a first optical switch that sequentially switches wavelengths among the respective input wavelengths into the one source fiber, a second optical switch that changes the at least one source between the at least two source fibers, a probe that does not touch the wound during use, the probe including the at least two source fibers and first and second detectors spaced thereon, and a processing unit that provides at least four independent measurements for calculation of an absorption coefficient μ_{α} and a scattering coefficient μ′ₛ, whereby the progress of the healing of the wound over time may be determined from changes in the absorption coefficient μ_{α} and the scattering coefficient μ′ₛ.

In the first embodiment of a non-contact probe configuration, the probe receives beams of input wavelengths from the at least one diode laser source via at least one of the at least two source fibers. The probe comprises a cube beamsplitter and a relay lens that together focus the beams on the wound. The relay lens and the cube beamsplitter are preferably configured such that scattered light from the wound returns to the first and second detectors through the relay lens and the cube beamsplitter. A CCD camera may be positioned to image the incident light on the surface of the wound through the relay lens and cube beamsplitter.

In a second embodiment of a non-contact probe configuration, the at least one diode laser source provides respective input wavelengths into a single source fiber and a single optical switch sequentially switches wavelengths among the respective input wavelengths into the single source fiber. In this embodiment, the non-contact probe includes the source fiber and four detectors spaced thereon, where the probe receives beams of input wavelengths from the at least one diode laser source via the source fiber. As in the first embodiment, the probe comprises a cube beamsplitter and a relay lens that together focus the beams on the wound.

Applications of such a non-contact device includes assessment of wound healing, pressure sores, ischemia for various diseases and their complications. The device also may be used for chronic wound healing and burn treatment, to evaluate the efficiency of hyperbaric oxygen treatments, or to evaluate the effectiveness of wound and burn gels, scaffolds, and other treatment modalities.

### BRIEF DESCRIPTION OF THE DRAWINGS

FIG. 1 illustrates a prior art frequency domain NIR device that uses a contact probe to measure the scattering coefficient μₛ′ and absorption μ_{α}.

FIG. 2 illustrates a typical experimental setup for a contact probe that contacts the wound.

FIG. 3 illustrates a frequency domain 2×2 NIR device that is used with a non-contact probe to measure the scattering coefficient μₛ′ and absorption μ_{α}.

FIG. 4 illustrates the non-contact probe for use with the NIR device of FIG. 3.

FIG. 5 illustrates an embodiment of a non-contact probe for accurately assessing the source/detector distance using non contact optics.

FIG. 6 illustrates a sample algorithm for determining the scattering coefficient and the absorption coefficient from the values measured by the device of FIGS. 3 and 4.

FIG. 7 illustrates another embodiment of an optical system for optical coupling in the embodiment of FIG. 3.

FIG. 8 illustrates plots that demonstrate the experimental results of light intensity and phase shift obtained from a silicon phantom, with both contact and non-contact devices.

### DETAILED DESCRIPTION OF ILLUSTRATIVE EMBODIMENTS

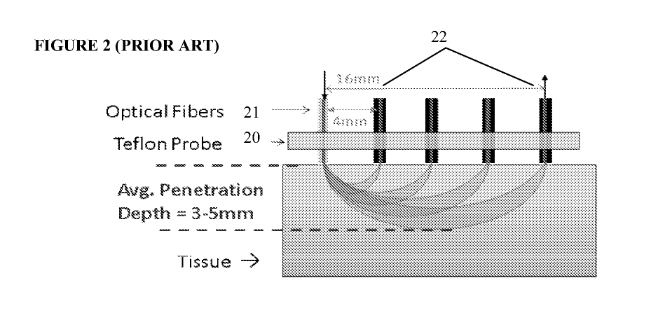

A detailed description of illustrative embodiments of the present invention will now be described with reference to FIGS. 2-8. Although this description provides a detailed example of possible implementations of the present invention, it should be noted that these details are intended to be exemplary and in no way delimit the scope of the invention.

The inventors have found that the method of Diffuse Near Infrared Spectroscopy (DNIS) allows the determination of the optical properties, specifically the absorption coefficient (μa) and scattering coefficient (μₛ′) of non homogeneous, strong light scattering media. Human and animal tissues can be analyzed quite accurately using the diffusion approximation of DNIS. Several non-invasive optical experimental techniques widely used for medical applications differ mainly by the type of incident light that illuminates the human or animal tissue. If frequency domain devices are considered where the incident light of NIR lasers is modulated by only one radio (RF) frequency, the absorption coefficient may be determined by measuring how the intensity of light scattering and the phase of registered light changes as a function of the distance between the light sources (mostly source fibers) and the detector fibers. The source and detector fibers are inserted in fixed positions on an experimental probe, usually made from a plastic semi-flexible material. A number of different combinations in source-detector distances is possible and gives rise to different device configurations, for example, 1 source—4 detectors, or 2 sources—2 detectors. In both of these cases, four experimental points are measured at four distinct source-detector separations. Typically, the probe is placed in full contact with the surface of tissue under investigation, which is illuminated through the source fibers. The required condition is that all fibers must be in contact with the tissue. The light registered by the detectors fibers consists of light that underwent multiple light scattering and as a result is propagating back to the surface; this corresponds to the semi infinite geometry of the diffusion approximation.

The typical experimental setup is shown in FIG. 2. As illustrated, the probe 20, which may be made of Teflon, for example, includes an optical fiber source 21 and 4 detector fibers 22. In such a configuration, the source light penetrates the tissue to an average depth of 3-mm, and the resultant light scattering is measured at the detector fibers 22, which are spaced at predetermined distances of, for example, 4 mm from each other.

However, as noted above, in several medical applications full contact between a probe and the surface of tissue is not a preferred mode of application, for example, in infectious wounds, in burns, or for measurements in during open surgery. The inventors have therefore developed a geometry for the Frequency Domain Device of DNIS where any contact between the fiber probe and the tissue is absent.

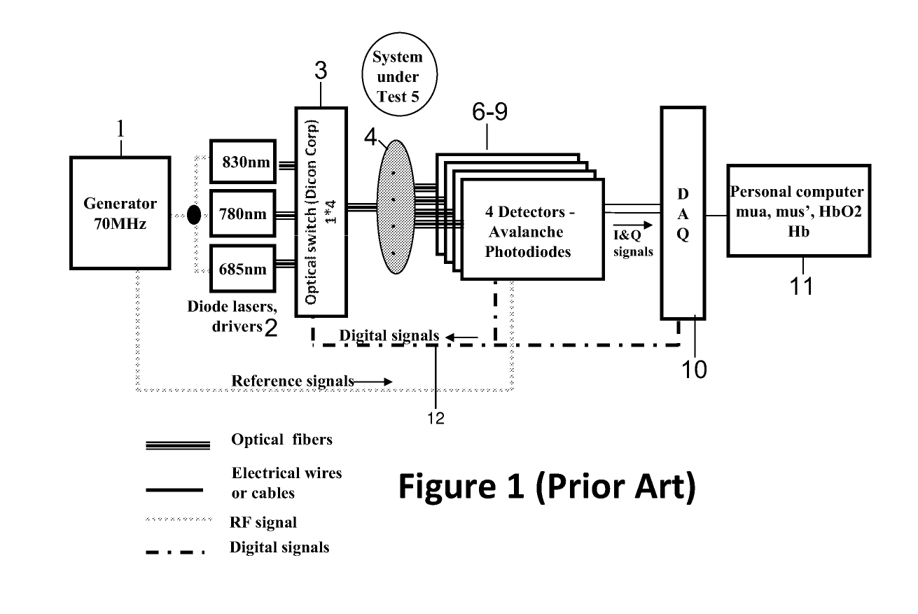

To design such a non-contact device, it is first desirable to accurately assess using non-contact optics the source/detector distance. In medical applications, the dependence of amplitude and phase on the source-detector separation ρ is fitted to theoretical formulas for calculating μₛ′ and μ_{α}. The accuracy of the determined optical properties depends on the precision of ρ. In traditional probes, the source/detector separation can be measured easily by different methods. In non-contact methods, on the other hand, the optical system transfers the image of skin to the plane of the detector fibers and also focuses the incident light on the skin surface. In order to overcome this problem, the detector fibers are placed in a special holder similar to the probe of FIG. 1 or 2, with known distances ρ. It is necessary to know the magnification of the relay lens with high accuracy because only then will the distances on the skin corresponding to the distances observed on the image plan be known. The relay lenses work in conditions that are very close to 1:1 in imaging applications. Those skilled in the art will appreciate that since the optical magnification depends on the relative position of the elements in the system, magnification may be changed to obtain additional experimental data at different source/detector distances in real time. Such changes may require different probes in a traditional contact registration system.

A non-contact device also needs a method for capturing true scattered light while minimizing artifacts such as stray beams. This is the case because the power of scattered light is very small and any stray beam can insert serious error into any results. Reflected beams from the skin surface can practically go in any direction because the surface of the skin is a rough surface and diffuse reflection with a range of reflection angles close to 180 degrees may be obtained.

In most clinical applications, the gap between the traditional probe and the skin is very small because the probe presses onto the skin with small pressure. Therefore, the light reflected from the spot of incident light cannot go back to the detector fibers. In order to minimize stray beams, the relay lens must be used as the main element of the optical system because lens construction limits and attenuates the stray beams stronger compared to usual lens systems.

Frequency Domain Device with Two Sources—Two Detectors

Current trends in creating new electronics and optoelectronics devices focus on the design and assembly of more compact yet highly reliable instruments as compared to ones available in the market several years ago. As particular optoelectronic elements become rather inexpensive and miniaturized, the cost effectiveness of such small devices is now attractive for mass deployment.

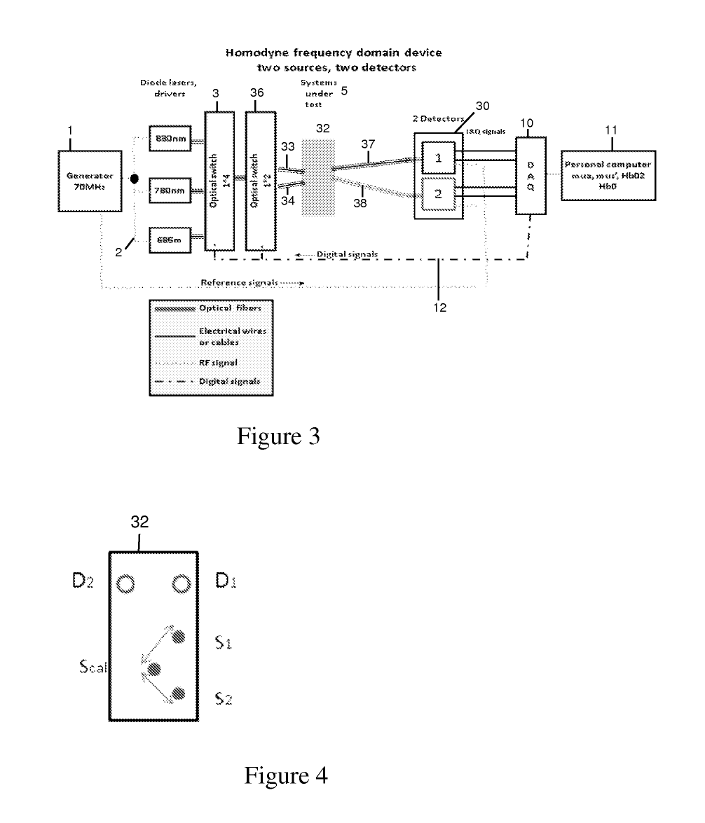

An exemplary embodiment of a 2×2 non-contact device in accordance with the invention is illustrated in FIG. 3. The illustrated device is small enough to be placed in an 8″ rack (not shown). The main components of the device are a 70 MHz modulation frequency generator 1, three NIR laser-diodes (685, 780, and 830 nm) 2, and two avalanche photo-diodes (APD) for simultaneous registration of the scattered light at four source-detector separations. In order to save space in the device, two detectors 30 are used. The detector modules 30 have relatively large sizes (3 inches by 2 inches); therefore, decreasing the amount of detectors from 4 to 2 saves considerable space and weight in the device. However, since it is still desirable to obtain four independent experimental measurements at four different source-detector separations, it is necessary to increase the number of source positions by a factor 2. Therefore, two sources are used in the probe 32, including two optical fibers 33, 34 as illustrated in FIG. 3. As shown in FIG. 4, S₁ and S₂ are the positions of the first and second source fibers, and D₁ and D₂ are the positions of the first and second detectors in the probe 32 during a typical measurement set up. S_{cal} is the position of first and second sources during instrument calibration. Arrows in FIG. 4 show placement of the first and second sources for calibration.

Necessary changes of the two source-two detector device versus the device of FIG. 1 extend also to the design configuration of the optical switches. The 4*1 prism optical switch 3 of FIG. 1 sequentially switches wavelengths among the different diode-lasers in the source fiber. A second 1×2 optical switch 36 is added in the 2-source-2-detector device of FIG. 3 in order to change the wavelength of the source between the two source fibers 33, 34. As a result, in the case of the probe 32, four source-detector separations (S₁D₁; S₁D₂; S₂D₁, S₂D₂) exist and are measured, providing four independent measurements.

The measured light amplitude and phase shift consist of both instrument and sample contributions. The amplitude obtained in each channel depends on the transmission of the optical fibers, the sensitivity of the avalanche photodiode, the gain of each detector block and the coupling of the fibers. The phase shift may be different in each channel because the optical and electrical signal delay depends on fiber length and coupling, length of RF coaxial cables, and delays in the detector circuits. Instrument calibration is designed to allow for separate variability due to the instrument hardware components from sample and measurement variability.

The probe 32 shown in FIG. 4 is used to obtain tissue measurements and also for the first instrument calibration. During measurements, the two detector fibers 37, 38 and the two source fibers 33, 34 are inserted in a Teflon probe at positions S1; S2; D1; D2. During calibration, the S1 fiber is placed in the S_{cal} position which is equidistant to D1 and D2 positions, yielding the same S_{cal}D1=S_{cal}D2 source-detector separations. The probe 32 is placed inside an intralipid solution (simulating infinite geometry) or on the liquid surface of the intralipid solution (simulating semi-infinite geometry). The detector areas are assumed to be very small so that the fluence rate does not change essentially over the surface of the detector fibers. Ideally, the phase shift should be identical for all detectors and the amplitudes should remain constant, for the distances S_{cal}D1=S_{cal}D2 and the fluencies are the same at positions D1 and D2. However, they are not. The difference in measured amplitude A_{cal 1} and phase Θ_{cal1} by the first and second detectors reflects the different response of each detector channel at the same fluence of scattered light because the source-detector separation is the same. The same procedure takes place for the second source with coefficients A_{cal2} and phase Θ_{ca12}. In principle, both sets of coefficients must be the same and the measured differences in experimental amplitudes and phases reflect in both cases the different power and different phase of modulation of the light sources. The above described calibration must be performed at all wavelengths, n. The measured experimental data are always corrected using an amplitude correction and a phase shift correction using the two sets of coefficients obtained (A_{cal1}Θ_{cal1}A_{cal2}Θ_{cal2}).

The non-contact theory derives an expression for the fluence Φ(\vec{r}) as a function of effective distances r_{b} and r₁, which depend on the Fresnel reflection at the tissue-air interface, the transport mean free path l* and the source-detector separation ρ along the sample surface.

$$Φ ( ρ , t ) = {S}_{ac} {exp}^{( - i ω t )} ( \frac{{e}^{( i {kr}_{1} )}}{{r}_{1}} - \frac{{e}^{( i {kr}_{b} )}}{{r}_{b}} ) \qquad ( 2 )$$

where k=kᵣₑₐₗ+ik_{imag} is a complex diffuse wavenumber. For measurements on tissue surfaces, r₁ and r_{b} can be written as:

$${r}_{1} = \sqrt{{μ}_{s}^{′ - 2} + {ρ}^{2}} ; \\ {r}_{b} = \sqrt{{( \frac{4}{3 {μ}_{s}^{′}} * \frac{( 1 + {R}_{eff} )}{( 1 - {R}_{eff} )} + \frac{1}{{μ}_{s}} )}^{2} + {ρ}^{2}} ; \qquad ( 3 )$$

Here ρ is the distance between the source (at ρ=0) and the detector position. In the semi-infinite geometry, when the detector is not close to the isotropic point source ρ>3*l*, a simple linear relation can be written:

$$\begin{matrix}ln [ {ρ}^{2} * {A}_{att} ( ρ ) ] = - {k}_{real} * ρ + ln ( {A}_{0} ) \\ {Θ}_{lag} = {k}_{imag} * ρ + {Θ}_{0} \\ where & ( 4 ) \\ {k}_{real} = \sqrt{\frac{3}{2} {μ}_{α} {μ}_{s}^{′}} \sqrt{{[ 1 + {( \frac{ω}{V {μ}_{α}} )}^{2} ]}^{1 / 2} + 1} ; \\ {k}_{imag} = \sqrt{\frac{3}{2} {μ}_{α} {μ}_{s}^{′}} \sqrt{{[ 1 + {( \frac{ω}{V {μ}_{α}} )}^{2} ]}^{1 / 2} - 1} ; & ( 5 )\end{matrix}$$

Aₐₜₜ(ρ) is the experimental intensity of scattered light measured in mV (millivolts), Θ_{lag}(ρ) is the experimentally measured change of phase relative to the phase of the 70 MHz generator, and V is the velocity of light in the medium (tissue). The corrected (using the calibration coefficients) experimental values of amplitude and phase are fitted to equation (4) and allow calculation of kᵣₑₐₗ,k_{imag}. The algebraic relations (5) allow the calculation of the absorption coefficient r¹α and the scattering coefficient μ′ₛ.

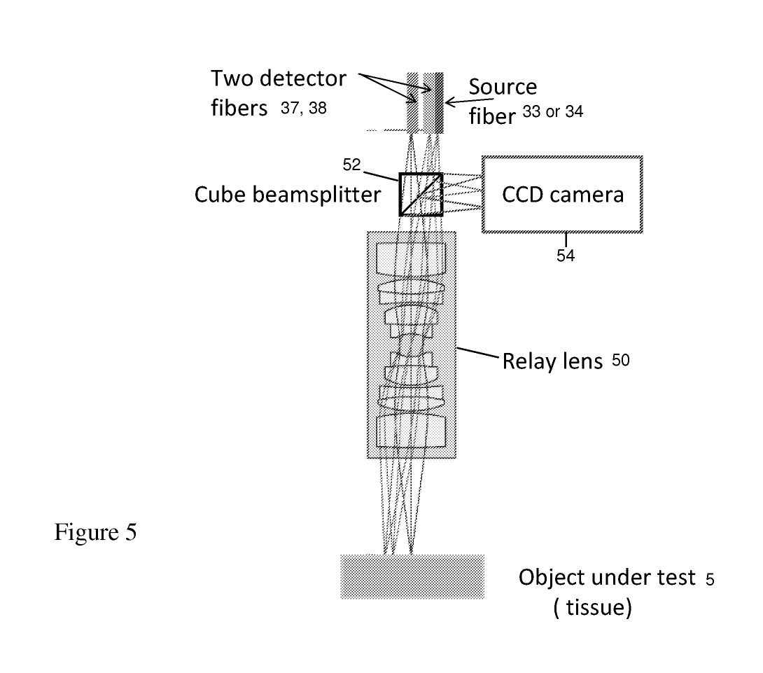

A detailed drawing of the optical parts of a first embodiment of a non-contact probe is shown in FIG. 5. The 2 source, 2 detector embodiment is represented by only one source 33 or 34 for purposes of simplifying the schematic of the path of beams in the optical system. Those skilled in the art will appreciate that the illustrated non-contact probe may also be used in the 1×4 source-detector arrangement of FIGS. 1 and 2. As illustrated, light is directed from the source fiber 33 or 34 which has a second end connected to the output of the optical switch 36 (FIG. 3). In the case where the numerical aperture (NA) of the source fiber is less than the NA of the relay lens 50, losses in the power of the incident light will be minimal. The light from the fiber will pass through the cube beamsplitter 52 (reflection of incident light is not shown) and the relay lens 50 will focus the beam on the tissue surface 5.

Scattering from the tissue 5 returns to the detection system through the relay lens 50. The Field Of View (FOV) of the relay lens 50 determines the size of the area that can be registered by the non-contact probe. The cube beam splitter 52 allows the light to be sent to the detector fibers 37, 38 that are placed in the focal plane of the relay lens 50. Using a CCD camera 54, the position of the incident light on the surface of the tissue 5 to be imaged can be followed along with the location of the image registration by the detector fibers 37, 38. During system calibration, silicone phantoms are used to determine the exact positioning of the optical components. All detector fibers 37, 38 can be placed in a common holder like that of FIG. 4 with the source fiber(s) 33, 34.

Those skilled in the art will appreciate that this machine vision measurement system offers a versatile solution for the non contact probe device. All optical elements can be designed and assembled by movable mounting flanges in one compact and reliable integrated system.

As noted above, in a non-contact probe the power of scattered light is very small and any stray beam can insert serious error in the obtained results. The existence of stray light in a contact probe is checked by measuring phantoms and volunteers, with and without a thin layer of immersion gel between the probe and the sample (phantom or skin). The immersion gel provides a medium of matching refractive index to the skin and is expected to decrease the intensity of reflected light by 20-40 times. In experiments, the obtained values of μ_{α} and μ′ₛ were identical, within experimental error±3-5% in the presence or absence of the immersion gel. This proves that the contact probe has minimal influence of stray beams because the intensity of reflected light depends strongly on Δn—the difference between the refraction indexes of both media at the boundary of incident light, I_{reflec}˜(Δn)² (Fresnel formulas). The immersion gel has a refractive index very close to the refractive index of skin and optical fibers. In order to minimize stray beams, a relay lens as the main element of the optical system should be used because lens construction limits and attenuates the stray beams stronger compared to usual system of objectives and lens systems.

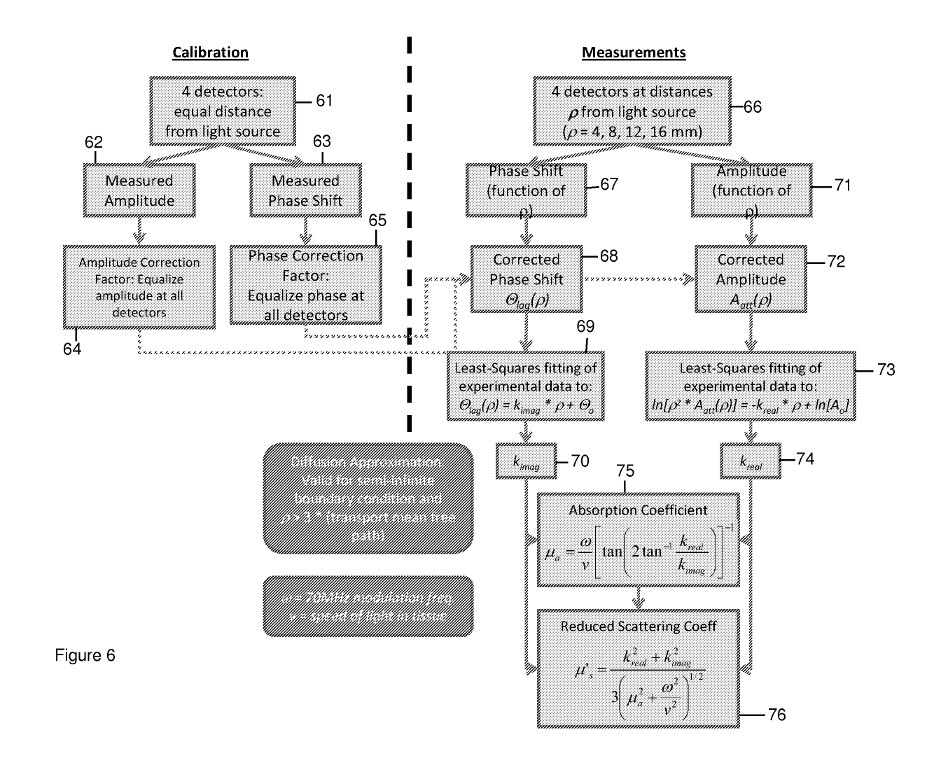

FIG. 6 illustrates a sample algorithm for determining the scattering coefficient and the absorption coefficient from the values measured by the device of FIGS. 3 and 4. As illustrated, the system is calibrated by placing 4 detectors at an equal distance from the light source at 61 and measuring the amplitude (62) and phase shift (63). The values are used to correct the amplitude and phase at all detectors at 64, 65. Then during measurements, the detectors are placed at distances ρ from the light source at 66, where ρ=4, 8, 12, 16 mm in an exemplary embodiment. The phase shift and amplitude (as a function of ρ) are corrected at steps 67-76 using the calibration factors. The values of k_{imag} and kᵣₑₐₗ are calculated at 70, 74, respectively, using the equations described above, and then the absorption and reduced scattering coefficients are calculated at 75, 76 using these values in the equations for absorption coefficient r′α and the scattering coefficient μ′ₛ as illustrated.

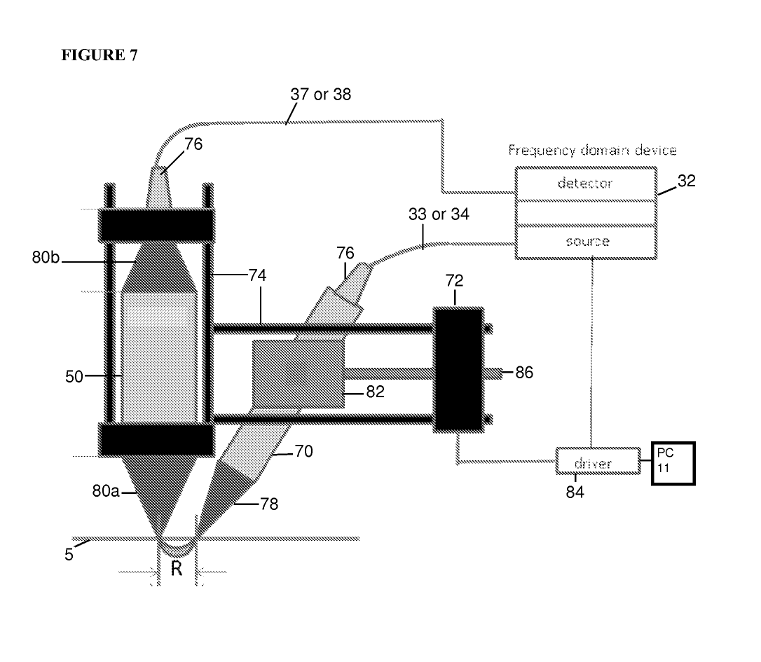

FIG. 7 illustrates another embodiment of an optical system for optical coupling in the embodiment of FIG. 3. As illustrated, this embodiment includes a relay lens 50 (available, for example, from Edmund optics having an effective focal length of 30 mm, f/#=4, object=image distances of 19.2 mm, and a length=48.9 mm) that transfers the image of the tissue surface 5 onto the detector fiber 37 or 38 (core diameter 1 mm in an exemplary embodiment). The choice of relay lens 50 is a key factor for determining the optimal performance of the optical system. The diameter of the core of the detector fibers determines the size of tissue surface (detector area) from which scattered light is collected by the detector. In this embodiment, the scattered light is registered by the same tissue surface 5 during all measurements, but the scattering volume is changing beneath the surface because the position of incident light (R-distances) is moving during the measurements, and therefore the penetration depth is altered.

In the optical embodiment of FIG. 7, the relay lens 50, the source system 70, and actuator 72 are mounted on a 30 mm cage system 74 from Thorlabs. The source system 3 may comprise a “Focus guide” (available from Fiberguide Industries with a focal length of 20 mm and a length of 50 mm) that is designed from several lenses and focuses the incident light from the source fiber 33 or 34 onto the tissue 5. The numerical aperture (NA) of the source fiber 33, 34 and of the optical system “Focus guide” must be similar for preserving the power of incident light. The source fiber 33 or 34 from the main block of the device is inserted into the FC fiber connector 76 of the optical fiber 33 or 34 in order to achieve reliable coupling. The cone of incident light 78 comes from the “Focus guide” 70, while the cones of scattered light 80a and 80b are registered by the device, where 80a is a true cone of light, while 80b is an image/mirror cone due to the lenses 50.

As illustrated in FIG. 7, the stepper actuator 74 moves the holder 82 of the “Focus guide” 70. Holder 82 includes a ball bearing stage (not shown) on which the “Focus guide” 70 is assembled. The stepper linear actuator 7 (available from Portescap Corporation) moves the source optical system 70 and consequently changes the position of the incident light spot (the top of cone 78) on the tissue surface 5 relative to the detector area (top of cone 80a). Software of the personal computer 11 (FIG. 3) controls the operation of actuator 7 by the application of TTL logic signals from driver 84. The axis 86 of the linear actuator 72 actually moves the optical system of the incident light. The source fiber 33 or 34 and detector fiber 37 or 38 transfer the incident and scattered light from the optical part to the main block 32 of the device. Two FC connectors 76 couple the incident light and the scattered light to the main device block 32.

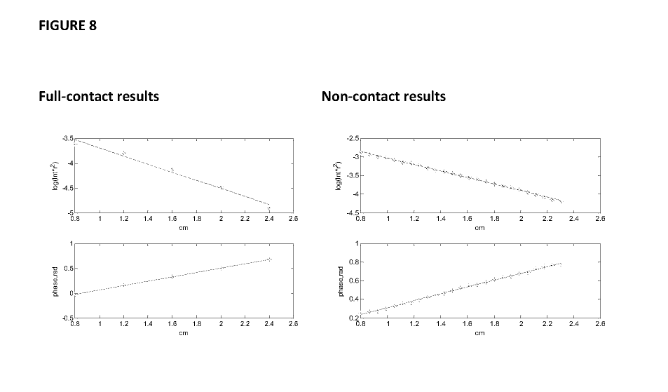

In the non-contact embodiment of FIG. 7, the number of experimental points is practically unlimited and depends from the duration of the experiment because it is possible to move the source light point at many small incremental distances relative to the detector area. The plots of FIG. 8 demonstrate the experimental results of light intensity and phase shift obtained from a silicon phantom, with both the contact and non-contact devices. As illustrated, the results obtained for the contact and non-contact devices are quite comparable. The large amount of experimental points in the “Non-contact configuration” allows one to obtain the optical properties of an object with higher accuracy in the fitting process as compared to any possible full-contact configuration.

Those skilled in the art also will readily appreciate that many additional modifications are possible in the exemplary embodiment without materially departing from the novel teachings and advantages of the invention. For example, the embodiments above contemplate 1 source—four detector and 2 source—2 non-contact detector configurations. Those skilled in the art that other configurations are certainly possible with appropriate adjustments in the optical system. Accordingly, any such modifications are intended to be included within the scope of this invention as defined by the following exemplary claims.

What is claimed:

1. A device for measuring the progress of healing of a wound over time, comprising:
at least one diode laser source that provides respective input wavelengths into at least one source fiber;
a first optical switch that sequentially switches wavelengths among the respective input wavelengths into said at least one source fiber;
a probe that does not touch the wound during use, said probe including said at least one source fiber and at least two detectors spaced thereon;
an optical system that provides source light from said at least one source fiber to the wound and that detects light scattered by the wound surface and provides the scattered light to said at least two detectors; and
a processing unit responsive to outputs of said at least two detectors for providing at least four independent measurements for calculation of an absorption coefficient μ_{α} and a scattering coefficient μ′ₛ, whereby the progress of the healing of the wound over time may be determined from said absorption coefficient μ_{α} and the scattering coefficient μ′ₛ.

2. A device as in claim 1, wherein said probe comprises two source fibers and two detectors and further comprising a second optical switch that changes the at least one laser diode source between said two source fibers.

3. A device as in claim 2, wherein the probe receives beams of input wavelengths from the at least one diode laser source via at least one of said two source fibers, and said optical system comprises a cube beamsplitter and a relay lens that together focus the beams on the wound.

4. A device as in claim 3, wherein the relay lens and the cube beamsplitter are configured such that scattered light from the wound returns to the two detectors through the relay lens, the cube beamsplitter, and two detector fibers.

5. A device as in claim 4, further comprising a CCD camera positioned to image the incident light on the surface of the wound through the relay lens and cube beamsplitter.

6. A device as in claim 1, wherein the optical system comprises a focus guide that receives beams of input wavelengths from the at least one diode laser source said at least one source fiber and focuses the light on a focal position of the wound and a relay lens that receives scattered light from the wound and provides the received scattered light to at least one of said at least two detectors via a detector fiber.

7. A device as in claim 6, further comprising a holder that holds the focus guide in place and a stepper actuator that moves the holder and focus guide to change the focal position of the input wavelengths on the wound.

8. A device as in claim 1, wherein said probe comprises one source fiber and four detectors.
